SEQUENCE LISTING (6 sequences)

SEQ ID NO: 1 - DNA, 1602 bp, Homo sapiens
atggcactgaggaatgtgccctttcgctcagaggtcctgggctgggaccccgacagccttgctgactatttcaagaagctcaactataaggactgtgagaaggcagtgaagaagtaccacatcgatggggctcgcttcttgaacctgacagaaaatgacatccagaagttccccaagctccgggtgccgattctcagtaagttaagtcaggaaatcaacaagaacgaagagaggaggagcatcttcacacgcaaaccccaagtcccgcggtttcctgaagagacagaaagccacgaagaggacaatgggggctggtcgtcctttgaagaagacgattatgaaagtcccaatgatgaccaggatggggaggatgatggagactatgagtcccccaatgaggaggaagaggcacccgtggaagatgacgcggattatgagccgccaccctccaatgacgaggaagctctgcagaactccatcctgcctgccaagcctttccccaactccaactccatgtacatcgaccggcccccctctgggaaaaccccccagcagcctcctgtgcccccccagagaccgatggccgccctcccgcccccaccagccggccggaatcactcgccactgcccccaccccagaccaaccacgaagaacccagcagaagcagaaaccacaaaacggcaaagctccctgctccttcaatagacagaagcacgaaacctcccctagatcgttcattagctccgtttgatagagaacccttcacactaggaaagaaaccaccattttctgacaagccctcgattccagcgggaaggtcactcggggagcatttacccaagattcaaaagcctcctttaccaccgaccacggaaagacatgaaaggagcagccccctgccagggaagaagccacctgtgccaaagcatggatggggaccagacagaagagagaatgatgaagatgatgtgcatcagagacctttgccccagccagcactacttcctatgagctccaacactttcccttcaagatctactaagccaagtcccatgaaccctctcccatcctctcacatgcctggagcattctcagaaagtaacagcagttttccacagagtgcctccctgccaccatacttctctcaaggccctagcaacagaccacctatcagagccgaaggcagaaacttccccttgccacttccaaacaaacctcggcccccatcccccgcggaggaagagaattcattaaatgaagagtggtacgtttcttatattacccgaccagaggcagaagctgctcttagaaagataaaccaggatggcacatttctggtcagagacagctctaaaaaaacaacaaccaatccatatgtcctcatggtgttgtacaaagataaagtttacaacatccagatccgttatcagaaggaaagtcaagtttacttgttgggaactggactccgagggaaagaggactttctgtctgtgtcagatattattgactacttcaggaaaatgccacttctgctcattgatgggaaaaaccgaggttccagataccagtgcacattaacgcatgctgcagggtacccatag

SEQ ID NO: 2 - protein, 533 aa, Homo sapiens
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 3 - protein, 737 aa, Homo sapiens
MPDHDSTALLSRQTKRRRVDIGVKRTVGTASAFFAKARATFFSAMNPQGSEQDVEYSVVQHADGEKSNVLRKLLKRANSYEDAMMPFPGATIISQLLKNNMNKNGGTEPSFQASGLSSTGSEVHQEDICSNSSRDSPPECLSPFGRPTMSQFDMDRLCDEHLRAKRARVENIIRGMSHSPSVALRGNENEREMAPQSVSPRESYRENKRKQKLPQQQQQSFQQLVSARKEQKREERRQLKQQLEDMQKQLRQLQEKFYQIYDSTDSENDEDGNLSEDSMRSEILDARAQDSVGRSDNEMCELDPGQFIDRARALIREQEMAENKPKREGNNKERDHGPNSLQPEGKHLAETLKQELNTAMSQVVDTVVKVFSAKPSRQVPQVFPPLQIPQARFAVNGENHNFHTANQRLQCFGDVIIPNPLDTFGNVQMASSTDQTEALPLVVRKNSSDQSASGPAAGGHHQPLHQSPLSATTGFTTSTFRHPFPLPLMAYPFQSPLGAPSGSFSGKDRASPESLDLTRDTTSLRTKMSSHHLSHHPCSPAHPPSTAEGLSLSLIKSECGDLQDMSEISPYSGSAMQEGLSPNHLKKAKLMFFYTRYPSSNMLKTYFSDVKFNRCITSQLIKWFSNFREFYYIQMEKYARQAINDGVTSTEELSITRDCELYRALNMHYNKANDFEVPERFLEVAQITLREFFNAIIAGKDVDPSWKKAIYKVICKLDSEVPEIFKSPNCLQELLHE

SEQ ID NO: 4 - protein, 162 aa, Homo sapiens
MWKVSALLFVLGSASLWVLAEGASTGQPEDDTETTGLEGGVAMPGAEDDVVTPGTSEDRYKSGLTTLVATSVNSVTGIRIEDLPTSESTVHAQEQSPSATASNVATSHSTEKVDGDTQTTVEKDGLSTVTLVGIIVGVLLAIGFIGAIIVVVMRKMSGRYSP

SEQ ID NO: 5 - DNA, 1908 bp, Homo sapiens
atggccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaa

SEQ ID NO: 6 - protein, 635 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

(19) United States
(12) Patent Application Publication
(10) Pub. No.: US 2023/0313197 A1
(43) Pub. Date: Oct. 5, 2023

(54) IN VIVO LYMPHOVENOUS ANASTOMOSIS

(71) Applicant: THE TRUSTEES OF INDIANA UNIVERSITY, Bloomington, IN (US)

(72) Inventors: Chandan K. SEN, Indianapolis, IN (US); Aladdin HASSAINEIN, Carmel, IN (US); Mithun SINHA, Indianapolis, IN (US)

(21) Appl. No.: 18/040,834

(22) PCT Filed: Aug. 31, 2021
(86) PCT No.: PCT/US2021/048404

§ 371 Date: Feb. 7, 2023

Related U.S. Application Data
(60) Provisional application No. 63/074,890, filed on Sep. 4, 2020.

(51) Int. Cl. C12N 15/113; C12N 15/52; A61P 7/00

(52) U.S. Cl. CPC C12N 15/1137; C12Y 207/10002; C12N 15/52; A61P 7/00; C12N 2310/14

(57) ABSTRACT

Disclosed herein are compositions and in vitro and in vivo methods for reprogramming lymphatic tissue to induce the lymphatic tissue to form a lymphovenous anastomosis with an adjacent vessel of the venous system.

### CROSS REFERENCE TO RELATED APPLICATIONS

This application claims priority to U.S. Provisional Patent Application No. 63/074,890 filed on Sep. 4, 2020, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 24 kilobytes ACII (Text) file named “342974_ST25.txt,” created on Aug. 24, 2021.

### BACKGROUND

Lymphedema refers to swelling of limbs due to accumulation of lymphatic fluid and is caused by the removal of or damage to lymph nodes. Lymphedema most commonly results from treatment of cancer although it can occur as the result of a rare congenital condition called primary lymphedema. Lymphedema usually occurs after cancer surgery or due to radiation therapy and is a chronic debilitating condition that affects around 250 million people worldwide. There is no current treatment that cures lymphedema.

The condition is primarily managed through surgical interventions of lymphovenous anastomosis (LVA) which relies on fusing the lymphatic vessels with the veins at the affected site. This helps to drain the lymphatic fluid back to the circulatory system. The technique depends on the identification of lymphatic channels, most commonly through imaging with indocyanine green (ICG) and fluorescent imaging. However, there are addition techniques that have been employed for the identification of the lymphatic channels including computed tomography (CT), lymphoscintigraphy, and magnetic resonance lymphangiography.

Once a suitable channel is identified, a recipient vein is necessary for creating an anastomosis between the two using supermicrosurgical techniques. With the use of ICG lymphangiography, the lymphatics are readily visualized; however, the recipient veins and venules can also be identified not by fluorescence but as a shadow crossing over the lymph vessels. Other techniques that have been described for localization of the veins include ultrasonography or echography.

Given the submillimeter size of the lymphatic vessels and recipient veins, the anastomosis presents unique challenges. The lymphatic channel, in particular, is often smaller than the vein, and the walls of the lymph vessels are especially thin and often collapse on themselves, making it difficult to place the needle into the lumen.

Accordingly, there is a need for a therapeutic methods and agents that induce localized lymphangiogenesis in a subject deficient in lymphatic function. Such lymphangiogenesis can be used to prevent or treat lymphedema. The present disclosure provides methods using targeted therapy to induce localized lymphangiogenesis to prevent/treat lymphedema, optionally through the use of tissue nanotransfection technology. As disclosed herein, localized lymphangiogenesis can also be used to create an anastomosis between lymph vessels and recipient veins/venules that avoids the difficulties encountered with microsurgery. As disclosed herein tissue nanotransfection (TNT) technology is used as a method of inducing native tissues to form a lymphovenous shunt.

### SUMMARY

There is no cure for lymphedema. The condition is managed through conservative (e.g. compression) and surgical interventions. Surgical intervention is by lymphovenous anastomosis (LVA) in which <1 mm lymphatic vessels are microsurgically connected with veins.

In accordance with one embodiment of the present disclosure compositions and methods of increasing localized lymphangiogenesis in a subject in need thereof is provided. In accordance with one embodiment compositions comprising lymphangiogenesis agents, including for example nucleic acids encoding for bioactive peptides, are formulated as pharmaceutical compositions. In one embodiment a composition comprising nucleic acid sequences encoding for gene products that increase in vivo Prox 1 activity is formulated as a composition suitable for transfection into skin tissue. In one embodiment the nucleic acids of the lymphangiogenesis compositions disclosed herein are transfected into skin tissue through the use of tissue nanotransfection (TNT). In one embodiment a method of treating a patient deficient in lymphatic function is provided wherein a lymphangiogenesis cocktail is transfected into skin cells to increase localized lymphangiogenesis. In one embodiment the lymphangiogenesis cocktail comprises a nucleic acid encoding for Prox 1. In one embodiment the lymphangiogenesis cocktail comprises a nucleic acid encoding for one or more proteins selected from the group consisting of Prospero homeobox protein 1 (Prox 1), SH2 domain-containing leukocyte protein (Slp-76) and Podoplanin (Pdpn). In one embodiment a non-viral vector is provided that comprises a nucleic acid sequence encoding Prox 1, wherein the nucleic acid sequence is operably linked to regulatory sequences that allow for expression of the encoded gene product in a mammalian cell. In one embodiment the regulatory sequences include a eukaryotic promoter, optionally wherein the promoter is a heterologous promoter. In accordance with one embodiment nucleic acids of the lymphangiogenesis cocktail are introduced into the cytosol of skin tissue cells in vivo, via nanotransfection (TNT), more particularly using a TNT device as described in Example 1 and shown in FIGS. 2A-2C.

As disclosed herein native tissues can be induced to form LVAs and thus avoid the need for microsurgical intervention. During fetal development the lymphatic vessels develop from veins. Once the lymphatic vessels are formed, the expression of two genes, SH2 domain-containing leukocyte protein 76 (Slp-76) and spleen tyrosine kinase (Syk) are activated which ensures that lymphatic vessels stay separate from veins. Applicant has discovered that if the expression of these two genes is down regulated, then lymphatic vessels fuse back to veins. The process can be further expedited by increasing the growth of lymphatic vessels, optionally through delivery of the Prox 1 gene to lymphatic vessel cells to increase lymphangiogenesis. The genetic cocktail delivered through TNT results in increased lymphatics and lymphovenous connections which eventually helps in draining the accumulated lymph fluid.

In accordance with one embodiment of the present disclosure, lymphangiogenesis and/or LVA formation is stimulated physiologically in vivo without surgical intervention using TNT technology by silencing genes (Slp-76 and Syk) and increasing expression of Prox 1 gene in a patient. The present procedures represent the first successfully in vivo induced lymphangiogenesis and LVA without surgical intervention.

In accordance with one embodiment a composition comprising nucleic acid sequences encoding for gene products that decrease in vivo Slp-76 and Syk activity and optionally increase in vivo Prox 1 activity is provided. In one embodiment the gene products that decrease Slp-76 and Syk activity comprise iRNA or other factors that inhibits the expression of the native Slp-76 and Syk genes. In one embodiment the nucleic acid sequence that increases Prox 1 activity is a nucleic acid sequence encoding for Prox 1. In one embodiment a non-viral vector is provided that comprises a first nucleic acid sequence encoding an inhibitor of Slp-76 activity, a second nucleic acid sequence encoding an inhibitor of Syk, and a third nucleic acid sequence encoding Prox 1, wherein the first, second, and third nucleic acid sequences are operably linked to regulatory sequences that allow for expression the encoded gene products in a mammalian cell. In one embodiment the non-viral vector comprises a single eukaryotic promoter operably linked to a multiple coding sequence that comprises said first, second, and third nucleic acid sequences wherein said multiple coding sequence further comprises internal ribosome entry sites present before each of the first, second, and third nucleic acid sequences. In one embodiment the eukaryotic promoter is a heterologous promoter.

In accordance with one embodiment a method is provided for enhancing the ability of lymphatic vessels to fuse to adjacent vessels of the venous system and form lymphovenous anastomosis in a targeted localized area. The method comprises the steps of introducing a cocktail of genes encoding for one or more inhibitors of Slp-76 and Syk and optionally a gene encoding for Prox 1 to the localized area where lymphovenous anastomosis is desired. In one embodiment the gene cocktail is delivered into the cytosol of cells of the target tissue through the use of TNT. In one embodiment cells of the lymphatic system are transfected with nucleic acid sequences that express, or induce the expression of, inhibitors of Slp-76 and/or Syk activity and/or a gene encoding Prox 1 within the transfected cells.

In one embodiment lymphatic tissue is transfected with a first nucleic acid sequence encoding an inhibitor for Slp-76, a second nucleic acid sequence encoding an inhibitor for Syk, and a third nucleic acid sequence encoding for Prox 1, wherein each of said first, second, and third nucleic acid sequences are operably linked to regulatory sequences that allow for expression (transcription and translation) of the gene products in the transfected cells. In accordance with one embodiment skin tissue, optionally lymphatic tissue, is transfected with a composition comprising the first, second, and third nucleic acid sequences, optionally wherein each of said first, second, third and fourth nucleic acid sequences are provided on separate plasmids. In one embodiment lymphatic tissue is transfected with a composition comprising the first, second, and third nucleic acid sequences wherein two or more of the first second, and third nucleic acids are located on a single plasmid, and in one embodiment all three of the first, second, and third nucleic acid sequences are located on a single plasmid.

In accordance with the present disclosure the target lymphatic tissue can be transfected with any of the reprogramming cocktails disclosed herein using any transformation technique known to those skilled in the art. In accordance with one embodiment nucleic acids of the reprogramming cocktail are introduced into the cytosol of lymphatic cells in vivo, via nanotransfection (TNT), more particularly using the TNT device described in Example 1 and shown in FIGS. 2A-2C.

In accordance with one embodiment a method is provided for preventing or minimizing lymphedema, or treating established lymphedema by targeting lymphatic tissue with reprogramming cocktails (e.g., PROX-1 plasmid) as described in Example 2 and shown in FIGS. 5A-5B.

In accordance with one embodiment a kit is provided for conducting in vivo transfection of lymphatic tissue and inducing the cells of the lymphatic tissue to form an LVA with an adjacent vessel of the venous system. In one embodiment the kit comprises a disposable nanotransfection device and a reprogramming cocktail. In one embodiment the nanotransfection device comprises a hollow microneedle array with one or more compartments for receiving a reprogramming cocktail solution or a cartridge comprising the reprogramming cocktail. In one embodiment the hollow microneedle array comprises an electrode (i.e., cathode, optionally gold-coated or silver-coated) that is positioned for contact with a solution loaded into the compartment of the device and a needle counter-electrode (i.e., anode) positioned for insertion intradermally into a patient's skin. In one embodiment the reprogramming cocktail solution comprises a first nucleic acid sequence encoding an inhibitor for Slp-76, a second nucleic acid sequence encoding and inhibitor for Syk, and a third nucleic acid sequence encoding for Prox 1. In one embodiment the inhibitor is an encoded iRNA specific for Slp-76, and Syk, respectively. In accordance with one embodiment the nanotransfection device is preloaded with the reprogramming cocktail solution.

### BRIEF DESCRIPTION OF THE DRAWINGS

FIG. 1 provides a schematic view of a TNT process based on a chip with a hollow microneedle array, conducted on exfoliated skin, and mediated by the hollow microneedles. A plasmid DNA solution (5) is retained in a reservoir (1) and is in fluid communication with a plurality of microneedles (2) of the hollow microneedle array. The plasmid DNA solution (5) is delivered to the skin tissue, comprising the epidermis (3) and dermis (4) layers, under a square electric pulse applied at microsecond level.

FIGS. 2A-2C provide schematics of the TNT chips with various nanochannels and microneedle arrays. FIG. 2A demonstrates a Type I hollow microneedle array with flat tip. FIG. 2B demonstrates a Type II hollow microneedle array with sharp tip and centered bore. FIG. 2C demonstrates a Type III hollow microneedle array with sharp tip and off-centered bore. Cross-sectional views are also shown for each type of TNT chip.

FIG. 3 provides details regarding the TNT process for delivering protein encoding sequences into cells of a mouse using a Type I hollow microneedle array with flat tip. More particularly nucleic acid sequences encoding for the protein Slp76 were delivered via TNT. The efficiency of cargo delivery using TNT was observed through FAM labeled DNA delivery on murine tail. The gene slp76 (NM_010696) was delivered in form of a plasmid comprising an open reading frame (ORF) for the slp76 gene. As shown in FIG. 3, increased expression of pro-lymphangiogenic gene slp76 was observed 72 h post-TNT, as measured through quantitative real time PCR.

FIGS. 4A-4C provide data showing an increased expression of lymphangiogenic gene products during resolution of tail lymphedemia. Mice were subjected to excisional surgery in their tails to induce lymphedema and the expression of lymphangiogenic gene products was monitored after surgery. During the resolution phase of lymphedema, increased expression of lymphangiogenic genes was observed for Slp76 (FIG. 4A), Prox1 FIG. 4B) and Pdpn (FIG. 4C) at ≥75 days post-surgery as measured through quantitative real time PCR compared to normal tail, n=11 (mice), p<0.

FIGS. 5A-5C demonstrate the efficacy of TNT treatment as protective against lymphedema. FIGS. 5A & 5B are photographs of mouse tails treated with TNT sham versus TNT with nucleic acid sequences encoding for Prox-1. The TNT therapy significantly decreases the amount of swelling and minimizes lymphedema in the experimental lymphedema murine tail model. FIG. 5C is a graph demonstrating mouse tail volume remains near baseline in the TNT treated Prox-1 group whereas the sham group treated with TNT but containing no nucleic acids exhibits lymphedema with spontaneous improvement as standard for the animal model.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “Enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis Enhancers function to increase transcription from nearby promoters Enhancers, like promoters, also often contain response elements that mediate the regulation of transcription. Enhancers often determine the regulation of expression.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans receiving a therapeutic treatment either with or without supervision of physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

### Embodiments

In accordance with one embodiment of the present disclosure, a method of treating a patient deficient in lymphatic function is provided wherein tissues impacted by lymphatic dysfunction are contacted with a lymphangiogenesis cocktail under conditions that enhance cellular uptake of the reprogramming composition components. In one embodiment the lymphangiogenesis cocktail is transfected into skin cells to increase localized lymphangiogenesis. In accordance with one embodiment the method comprises transfecting tissues in close proximity to the region deficient in lymphatic function with nucleic acid sequences that enhance the activity of agents involved in lymphangiogenesis, including for example, increasing the activity of Prox 1. In one embodiment a lymphangiogenesis cocktail is introduced into the cytosol of cells of the afflicted tissue, wherein the cocktail comprises a nucleic acid encoding for Prox 1. In one embodiment the lymphangiogenesis cocktail comprises nucleic acid sequences encoding for one or more proteins selected from the group consisting of Prox 1, Slp76 and Pdpn. In one embodiment the cells of tissues exhibiting deficient lymphatic function are transfected with nucleic acid sequences that encode for a protein having at least 95% sequence identity to a protein selected from the group consisting of SEQ ID NO: 2, SEQ ID NO: 3 and SEQ ID NO: 4.

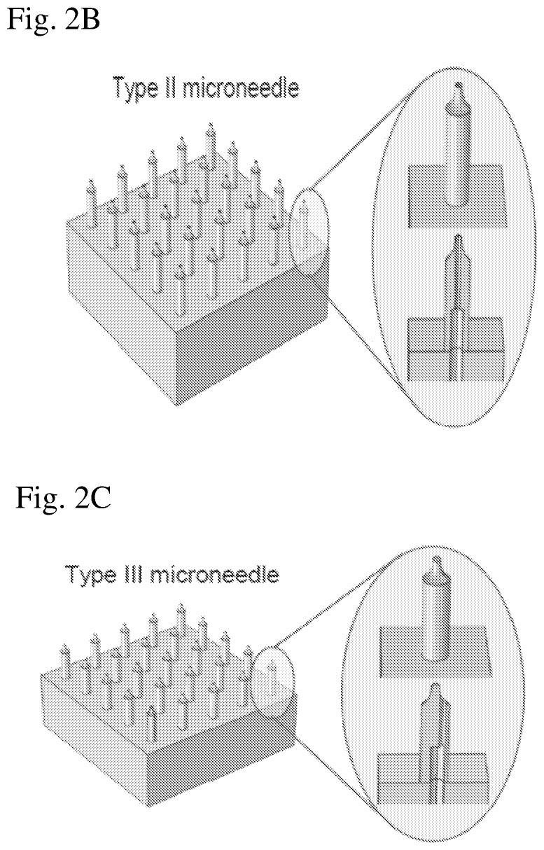

In one embodiment skin tissue associated with a region of the body (e.g. the legs) that is deficient in lymphatic function is transfected with nucleic acid sequences that enhance localized lymphangiogenesis. In one embodiment skin tissue are transfected with nucleic acids encoding for a protein having at least 95% sequence identity to a protein selected from the group consisting of SEQ ID NO: 2, SEQ ID NO: 3 and SEQ ID NO: 4. In one embodiment skin tissue is transfected with nucleic acids encoding for a protein having at least 95% sequence identity to SEQ ID NO: 3. In one embodiment any of the nucleic acids disclosed herein can be transfected into the cells of target tissue in vivo through the use of tissue nanotransfection (TNT). However alternative methods of transfection techniques known to those skilled in the art can also be used to transfect the nucleic acids of the present invention into cells. In one embodiment a non-viral vector is provided that comprises a nucleic acid sequence encoding Prox 1, wherein the nucleic acid sequence is operably linked to regulatory sequences that allow for expression the encoded gene product in a mammalian cell. In one embodiment the regulatory sequences include a eukaryotic promoter, optionally wherein the promoter is a heterologous promoter. In accordance with one embodiment nucleic acids of the lymphangiogenesis cocktail are introduced into the cytosol of skin tissue cells in vivo, via nanotransfection (TNT), more particularly using a TNT device as described in Example 1 and shown in FIGS. 2A-2C.

In another embodiment of the present disclosure compositions and methods are provided for transfecting tissues and cells in vivo to enhance the expression of Prox 1, and optionally inhibit the expression of Slp-76 and Syk in lymphatic tissues. In one embodiment a method of enhancing the formation of lymphovenous anastomosis (LVA) in a targeted localized region is provided to treat patients suffering from lymphedema. In one embodiment the method comprises transfecting tissues and cells in vivo to enhance the expression of Prox 1, and optionally inhibit the expression of Slp-76 and Syk in lymphatic tissues. In accordance with one embodiment a method of inducing lymphovenous anastomosis (LVA) formation in vivo in a subject with lymphedema is provided wherein target lymphatic tissue is reprogrammed in vivo to induce localized lymphangiogenesis and create an anastomosis between lymph vessels and recipient veins/venules. The method comprises contacting the cells of the target lymphatic tissue with a reprogramming composition under conditions that enhance cellular uptake of the reprogramming composition components. In one embodiment the reprogramming composition comprises
i) a first nucleic acid sequence encoding an iRNA that specifically binds to Syk nucleic acid sequences; or
ii) a second nucleic acid sequence encoding an iRNA that specifically binds to Slp-76 nucleic acid sequences; or
iii) a third nucleic acid sequence encoding for Prox 1; or
iv) a combination of i) and iii); or
v) a combination of ii) and iii); or
vi) a combination of i), ii) and iii).

In one embodiment the iRNA is a nucleic acid sequence of at least 10 nucleotides that specifically binds to the target gene or encoded mRNA of the respective identified genes. In one embodiment the iRNA that specifically binds to Syk nucleic acid sequences comprises a 10, 15, 20, or 30 contiguous nucleotide fragment of SEQ ID NO: 5 or complement thereof, and the iRNA that specifically binds to Slp-76 nucleic acid sequences comprises, 15, 20, or 30 contiguous nucleotide fragment of SEQ ID NO: 1 or complement thereof.

The polynucleotides of the present disclosure may be delivered to the lymphatic tissue via a gene gun, a microparticle or nanoparticle suitable for such delivery, a liposome or other membrane bound vesicle suitable for such delivery, injection of DNA or viral-based vectors, or transfection by electroporation, using a three-dimensional nanochannel electroporation, a tissue nanotransfection (TNT) device, or a deep-topical tissue nanoelectroinjection device. In some embodiments, a viral vector can be used. However, in other embodiments, the polynucleotides are not delivered virally.

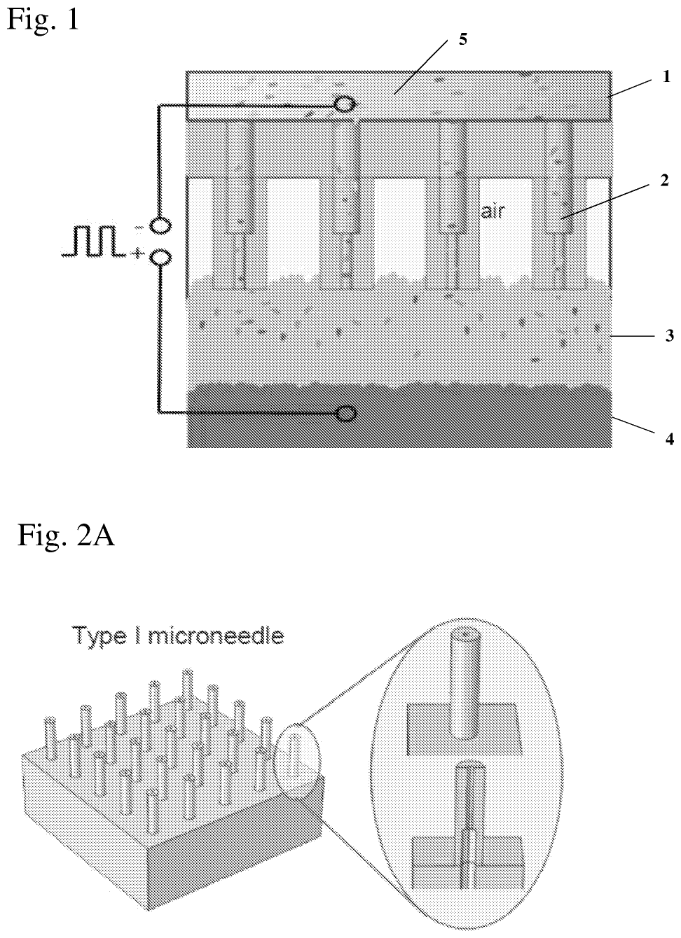

Electroporation is a technique in which an electrical field is applied to cells in order to increase permeability of the cell membrane, allowing cargo (e.g., reprogramming factors) to be introduced into cells (see FIG. 1). Electroporation is a common technique for introducing foreign DNA into cells. FIG. 2A-2C provide examples of microchannel and microneedle arrays that can be used to transfect somatic cells in vivo. Additional details regarding such devices have been described in U.S. patent application Nos. 62/903,298 and 62/877,060, the disclosures of which are expressly incorporated by reference.

Tissue nanotransfection allows for direct cytosolic delivery of cargo (e.g, reprogramming factors) into cells by applying a highly intense and focused electric field through arrayed nanochannels, which benignly nanoporates the juxtaposing tissue cell members, and electrophoretically drives cargo into the cells.

The compositions of the present invention may further comprise a pharmaceutical carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

TNT provides a method for localized gene delivery that causes direct conversion of tissue function in vivo under immune surveillance without the need for any laboratory procedures. By using TNT with plasmids, it is possible to temporally and spatially control expression of a gene. Spatial control with TNT allows for transfection of a target area such as a portion of skin tissue without transfection of other tissues.

As disclosed in greater detail in the Examples, a hollow needle array structure has been designed that enables efficient cutaneous delivery of loaded drugs including nucleic acid sequences. Three different types of silicon hollow needle arrays can be prepared for TNT applications (as shown schematically in FIGS. 2A-2C) with bore diameter ranging from nm to μm in sizes. In less than a second, the silicon hollow needle arrays disclosed herein enable delivery of active factors to specific depth in mouse, rat and human tissue.

In accordance with one embodiment a composition is provided for reprogramming cells and tissues, and more particularly reprogramming lymphatic tissues in vivo. In one embodiment the composition comprises a first nucleic acid sequence encoding for an inhibitor of Slp-76; a second nucleic acid sequence encoding for an inhibitor of Syk and; a third nucleic acid sequence encoding for Prox 1, wherein each of said first, second, and third nucleic acid sequences are operably linked to regulatory sequences for expression of the encoded proteins in eukaryotic cells, including mammalian cells. In one embodiment the composition comprises each of the first, second, and third nucleic acid sequences. In one embodiment the composition consists of the first, second, and third nucleic acids and a pharmaceutically acceptable carrier, optionally wherein each of the first, second, and third nucleic acid sequences are operably linked to a heterologous promoter.

Applicant has stimulated LVA physiologically in vivo without surgical intervention using TNT technology by silencing genes (Slp-76 and Syk) and increasing expression of Prox 1 gene in murine. This is the first approach which has successfully induced LVA without surgical intervention.

In one embodiment a method of transfecting cells of lymphatic tissues in vivo is provided, wherein said method comprises the step of:
delivering intracellularly into said cells of lymphatic tissue DNA comprising
a first nucleic acid sequence comprising Syk nucleic acid sequences;
a second nucleic acid sequence comprising Slp-76 nucleic acid sequences; and/or
a third nucleic acid sequence encoding for Prox 1.

In accordance with embodiment 1, a method for inducing lymphangiogenesis in vivo in a subject in need thereof is provided, wherein the method comprises the step of contacting skin tissue of said subject with a composition comprising a nucleic acid encoding for a gene product that increases the in vivo activity of a protein selected from the group consisting of Prospero homeobox protein 1 (Prox 1), SH2 domain-containing leukocyte protein (SLP-76) and Podoplanin (Pdpn) under conditions conducive for uptake of the composition components, optionally wherein the composition components are nucleic acids encoding products that induce lymphangiogenesis.

In accordance with embodiment 2 the method of embodiment 1 is provided wherein the subject's tissue (e.g., skin) is transfected with a nucleic acid sequence encoding for a protein selected from the group consisting of Prox 1, Slp76 and Pdpn.

In accordance with embodiment 3 the method of embodiment 1 or 2 is provided wherein skin tissue is transfected with a nucleic acid sequence encoding for Prox 1.

In accordance with embodiment 4 the method of any one of embodiments 1-3 is provided wherein said transfected nucleic acid encodes for a peptide having at least 95% sequence identity with the Prox 1 peptide sequence of SEQ ID NO: 3.

In accordance with embodiment 5 the method of any one of embodiments 1˜4 is provided wherein the transfection of skin tissue is via tissue nanotransfection.

In accordance with embodiment 6 a method of reprograming cells of lymphatic tissues to be more receptive to form a lymphovenous anastomosis (LVA) with an adjacent vessel of the venous system is provided, wherein the method comprises the step of delivering intracellularly into said cells of lymphatic tissue DNA comprising
a first nucleic acid sequence encoding an iRNA that specifically binds to Syk nucleic acid sequences;
a second nucleic acid sequence encoding an iRNA that specifically binds to Slp-76 nucleic acid sequences; and
a third nucleic acid sequence encoding for Prox 1.

In accordance with embodiment 7 the method of embodiment 6 is provided wherein one or more expression vectors are delivered intracellularly into said cells of the lymphatic tissue wherein said expression vectors comprise said first, second, and third nucleic acid sequences In accordance with embodiment 8 the method of any one of embodiments 6-7 is provided wherein said first, second, and third nucleic acid sequences are each delivered simultaneously into the cytosol of cells of said lymphatic tissue in vivo.

In accordance with embodiment 9 the method of any one of embodiments 6-8 is provided wherein two or more of said first, second, and third nucleic acids are part of an expression vector wherein the expression vector comprises a single eukaryotic promoter operably linked to a multiple coding sequence that comprises said two or more first, second, and third nucleic acid sequences wherein said multiple coding sequence further comprises internal ribosome entry sites present before each of said two or more first, second, and third nucleic acid sequences.

In accordance with embodiment 10 the method of any one of embodiments 6-9 is provided wherein each of said first, second, and third nucleic acids are located on a single expression vector.

In accordance with embodiment 11 the method of any one of embodiments 6-10 is provided wherein the intracellular delivery is via tissue nanotransfection.

In accordance with embodiment 12 a method of inducing lymphovenous anastomosis (LVA) formation in vivo in a subject with lymphedema is provided, wherein the method comprises the step of reprogramming targeted lymphatic tissue in vivo to produce reprogramed lymphatic tissue by
contacting the cells of said target lymphatic tissue with a reprogramming composition under conditions that enhance cellular uptake of the reprogramming composition components, wherein the reprogramming composition comprises
   a first nucleic acid sequence encoding an iRNA that specifically binds to Syk nucleic acid sequences;
   a second nucleic acid sequence encoding an iRNA that specifically binds to Slp-76 nucleic acid sequences; and
   a third nucleic acid sequence encoding for Prox 1.

In accordance with embodiment 13 the method of embodiment 12 is provided wherein the iRNA that specifically binds to Syk nucleic acid sequences comprises 10 contiguous nucleotides of SEQ ID NO: 5 or complement thereof, and the iRNA that specifically binds to Slp-76 nucleic acid sequences comprises 10 contiguous nucleotides of SEQ ID NO: 1 or complement thereof.

In accordance with embodiment 14 the method of any one of embodiments 12-13 is provided wherein the cellular uptake of the reprogramming composition components is induced via tissue nanotransfection.

In accordance with embodiment 15 the method of any one of embodiments 12-4 is provided wherein two or more of said first, second, and third nucleic acids are part of an expression vector wherein the expression vector comprises a single eukaryotic promoter operably linked to a multiple coding sequence that comprises said two or more of the first, second, and third nucleic acid sequences wherein said multiple coding sequence further comprises internal ribosome entry sites present before each of said two or more of the first, second, and third nucleic acid sequences.

In accordance with embodiment 16 the method of any one of embodiments 12-15 is provided wherein said multiple coding sequence comprises all three of said first, second, and third nucleic acid sequence, each proceeded by an internal ribosome entry sites and operably linked to said single eukaryotic promoter.

In accordance with embodiment 17 the method of any one of embodiments 12-16 is provided wherein the first, second, and third nucleic acid sequences are part of a non-viral vector.

In accordance with embodiment 18 a kit for conducting in vivo transfection of lymphatic tissue is provided, wherein said kit comprises
a disposable nanotransfection device; and
a reprogramming cocktail, wherein the reprogramming cocktail solution comprises a nucleic acid sequence encoding for Prox 1.

In accordance with embodiment 19 the kit of embodiment 18 is further provided with a nucleic acid sequence encoding an iRNA that specifically binds to Slp-76 nucleic acid sequences; and optionally a nucleic acid sequence encoding an iRNA that specifically binds to Syk nucleic acid sequences.

In accordance with embodiment 20 the kit of embodiment 18 or 19 is provided wherein the nanotransfection device comprises a hollow microneedle array with one or more compartments for receiving said reprogramming cocktail solution.

### Example 1

### Use of TNT to Deliver and Express Genes in a Murine Tail Model of Lymphedema

A plasmid vector comprising an Slp76 open reading frame was delivered into the cytosol of mouse skin tissues using TNT. 60 μg of the Slp76 encoding plasmid was loaded in the reservoir of a TNT device. Square wave pulsed electrical stimulation (10×10 ms pulses, 250V, 10 mA) was applied across the electrodes to drive the plasmid into the tissue through the arrayed nanochannels. Validation of TNT was assessed through increased expression of Slp76, 72 h post-TNT as measured through quantitative real time PCR.

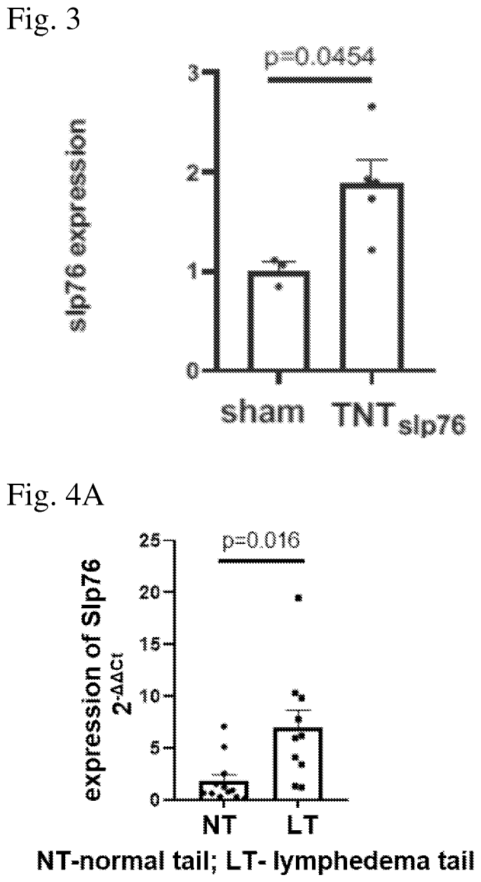

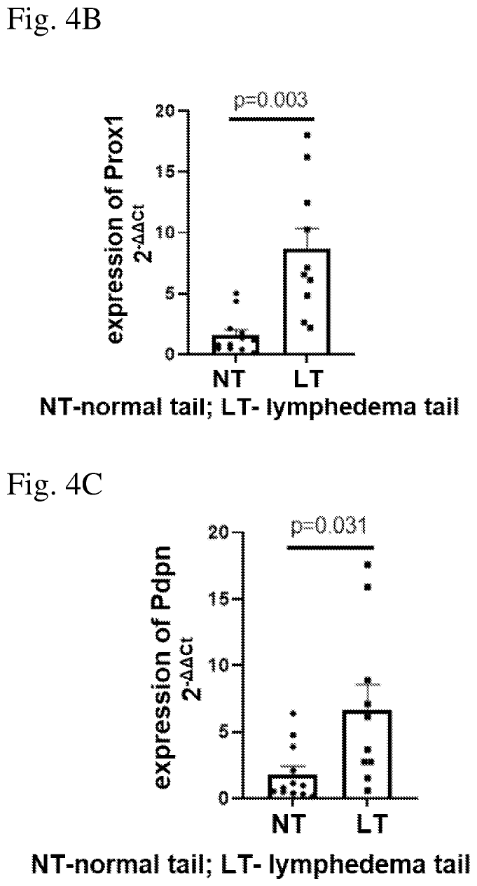

As shown in FIG. 3 and FIGS. 4A-4C applicant has demonstrated the ability to deliver nucleic acid sequences to mouse cells in vivo and to enhance expression of the encoded gene products.

### Example 2

### Enhanced Expression of Lymphangiogenic Gene Products During Resolution of Tail Lymphedemia

A murine model of lymphedemia was used to monitor gene expression after surgical induced lymphedema. Mice (n=11) were subjected to excisional surgery in their tails to induce lymphedema, and the expression of lymphangiogenic gene products was monitored post-surgery. As shown in FIGS. 4A-4C an increased expression of lymphangiogenic gene products was observed during resolution of tail lymphedemia. More particularly, increased expression of lymphangiogenic genes was observed for Slp76 (FIG. 4A), Prox 1 (FIG. 4B) and Pdpn (FIG. 4C) at ≥75 days post-surgery as measured through quantitative real time PCR compared to normal tail, n=11 (mice), p<0.

### Example 3

### Treatment of Lymphedema in a Mouse Tail Model System

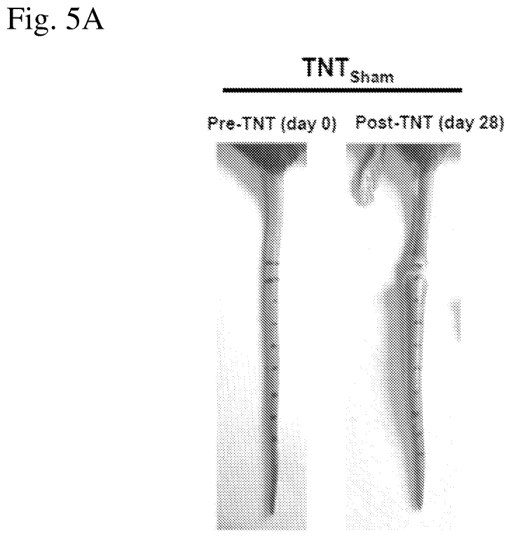

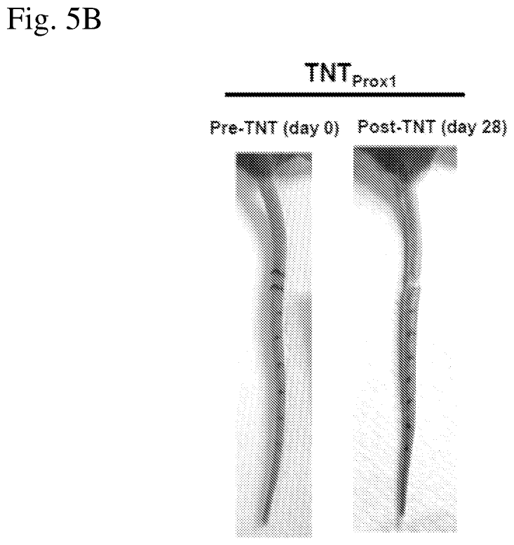

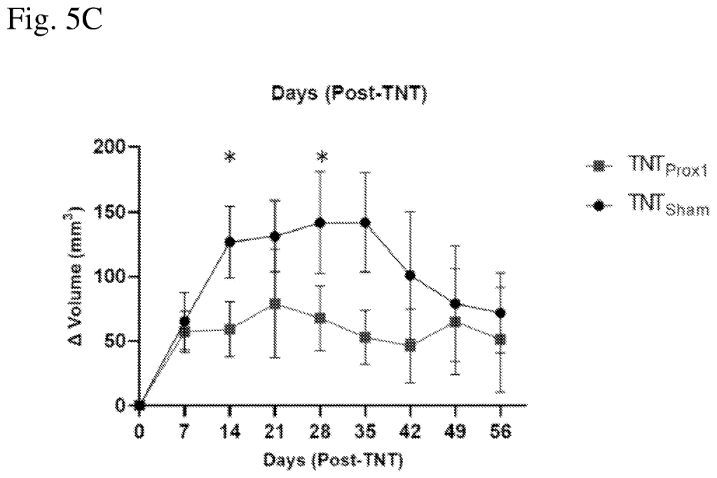

A well-described mouse-tail model was used to study the effect of introducing lymphangiogenic genes into the tissues impacted by surgical induced lymphatic dysfunction. Briefly, lymphatic stasis was induced by excising a 2-mm circumferential segment of skin and deep lymphatics 20 mm from the base of the tail. Plasmid DNA encoding for Prox1 (60 μg) was loaded in the reservoir of a TNT device and square wave pulsed electrical stimulation (10×10 ms pulses, 250V, 10 mA) was applied across the electrodes to drive the plasmid into the tissue through the arrayed nanochannels. A representative image of TNT sham control (devoid of Prox1 encoding sequences) treated mouse tails at day 0 and day 28 post-TNT is shown in FIG. 5A. A representative image of Prox1 TNT (TNT_{Prox1}) transfected mouse tails at day 0 and day 28 post-TNT is shown in FIG. 5B. The cohort of mice receiving TNT_{Prox1} didn't exhibit tail swelling associated with onset of lymphedema. A line graph depicting the progression of lymphedema in both groups over a period of 56 days (N=6 mice in each group) is shown in FIG. 5C, demonstrating the efficacy of the TNT_{Prox1} treatment.

1. A method for inducing lymphangiogenesis in vivo in a subject in need thereof, said method comprising the step of transfecting skin tissue of said subject with a composition comprising a nucleic acid encoding for a gene product that increases the in vivo activity of Prospero homeobox protein 1 (Prox 1).

2. The method of claim 1 wherein said skin tissue is transfected with a nucleic acid sequence encoding for Prox 1, and a nucleic acid encoding for a gene product that decreases the in vivo activity of a protein selected from the group consisting of SH2 domain-containing leukocyte protein (SLP-76) and Podoplanin (Pdpn).

3. The method of claim 1 wherein said skin tissue is transfected with a nucleic acid sequence encoding for Prox 1.

4. The method of claim 3 wherein said transfected nucleic acid encodes for a peptide having at least 95% sequence identity with SEQ ID NO: 3.

5. The method of claim 4 wherein the transfection of skin tissue is via tissue nanotransfection.

6. A method of reprograming cells of lymphatic tissues to be more receptive to form a lymphovenous anastomosis (LVA) with an adjacent vessel of the venous system, said method comprising the step of:
delivering intracellularly into said cells of lymphatic tissue DNA comprising
a first nucleic acid sequence encoding an iRNA that specifically binds to spleen tyrosine kinase (Syk) nucleic acid sequences;
a second nucleic acid sequence encoding an iRNA that specifically binds to Slp-76 nucleic acid sequences; and
a third nucleic acid sequence encoding for Prox 1.

7. The method of claim 6 wherein one or more expression vectors are delivered intracellularly into said cells of the lymphatic tissue wherein said expression vectors comprise said first, second, and third nucleic acid sequences.

8. The method of claim 6 or 7 wherein said first, second, and third nucleic acid sequences are each delivered simultaneously into the cytosol of cells of said lymphatic tissue in vivo.

9. The method of claim 7 wherein two or more of said first, second, and third nucleic acids are part of an expression vector wherein the expression vector comprises a single eukaryotic promoter operably linked to a multiple coding sequence that comprises said two or more first, second, and third nucleic acid sequences wherein said multiple coding sequence further comprises internal ribosome entry sites present before each of said two or more first, second, and third nucleic acid sequences.

10. The method of claim 9 wherein each of said first, second, and third nucleic acids are located on a single expression vector.

11. The method of claim 6 wherein the intracellular delivery is via tissue nanotransfection.

12. A method of inducing lymphovenous anastomosis (LVA) formation in vivo in a subject with lymphedema, said method comprising the step of reprogramming targeted lymphatic tissue in vivo to produce reprogramed lymphatic tissue by
contacting the cells of said target lymphatic tissue with a reprogramming composition under conditions that enhance cellular uptake of the reprogramming composition components, wherein the reprogramming composition comprises
a first nucleic acid sequence encoding an iRNA that specifically binds to Syk nucleic acid sequences;
a second nucleic acid sequence encoding an iRNA that specifically binds to Slp-76 nucleic acid sequences; and
a third nucleic acid sequence encoding for Prox 1.

13. The method of claim 12 wherein the iRNA that specifically binds to Syk nucleic acid sequences comprises 10 contiguous nucleotides of SEQ ID NO: 5 or complement thereof, and the iRNA that specifically binds to Slp-′76 nucleic acid sequences comprises 10 contiguous nucleotides of SEQ ID NO: 1 or complement thereof.

14. The method of claim 12 wherein the cellular uptake of the reprogramming composition components is induced via tissue nanotransfection.

15. The method of claim 12 wherein two or more of said first, second, and third nucleic acids are part of an expression vector wherein the expression vector comprises a single eukaryotic promoter operably linked to a multiple coding sequence that comprises said two or more of the first, second, and third nucleic acid sequences wherein said multiple coding sequence further comprises internal ribosome entry sites present before each of said two or more of the first, second, and third nucleic acid sequences.

16. The composition of claim 15 wherein said multiple coding sequence comprises all three of said first, second, and third nucleic acid sequence, each proceeded by an internal ribosome entry sites and operably linked to said single eukaryotic promoter.

17. The composition of claim 12 wherein the first, second, and third nucleic acid sequences are part of a non-viral vector.

18. A kit for conducting in vivo transfection of lymphatic tissue, said kit comprising
a disposable nanotransfection device; and
a reprogramming cocktail, wherein the reprogramming cocktail solution comprises
a first nucleic acid sequence encoding an iRNA that specifically binds to Syk nucleic acid sequences;
a second nucleic acid sequence encoding an iRNA that specifically binds to Slp-76 nucleic acid sequences; and
a third nucleic acid sequence encoding for Prox 1.

19. The kit of claim 18 wherein the nanotransfection device comprises a hollow microneedle array with one or more compartments for receiving said reprogramming cocktail solution.
